SEQUENCE LISTING (4 sequences)

SEQ ID NO: 1 - DNA, 13 bp, Artificial Sequence
  Synthetic DNA-puromycin linker/oligomer
ctacgatcggaaa

SEQ ID NO: 2 - DNA, 55 bp, Artificial Sequence
  Synthetic Camelid Consensus Sequence
aaagattacaaagatgatgatgataaaggaggaggaggaggaggaacaacggcag

SEQ ID NO: 3 - DNA, 20 bp, Artificial Sequence
  Synthetic PNA Oligonucleotide
ccgatcgtagctgccgttgt

SEQ ID NO: 4 - DNA, 20 bp, Artificial Sequence
  Synthetic DNA oligonucleotide
ccgatcgtagctgccgttgt

(19) United States
(12) Patent Application Publication
(10) Pub. No.: US 2014/0128275 A1
(43) Pub. Date: May 8, 2014

(54) COMPLEX OF NON-COVALENTLY BOUND PROTEIN WITH ENCODING NUCLEIC ACIDS AND USES THEREOF

(71) Applicant: Syndecion, LLC, Highland, MD (US)

(72) Inventors: Alan D. King, Highland, MD (US); Stephen B. Deitz, Catonsville, MD (US)

(73) Assignee: Syndecion, LLC, Highland, MD (US)

(21) Appl. No.: 13/971,509

(22) Filed: Aug. 20, 2013

Related U.S. Application Data
(63) Continuation-in-part of application No. 13/971,184, filed on Aug. 20, 2013.
(60) Provisional application No. 61/742,882, filed on Aug. 21, 2012.
(60) Provisional application No. 61/742,883, filed on Aug. 21, 2012.

(51) Int. Cl. C07K 17/10

(52) U.S. Cl. CPC C07K 17/10
USPC 506/9; 506/26

(57) ABSTRACT

A method of binding a protein to its encoding nucleic acid is disclosed wherein the method of binding is non-covalent at one or more locations between the protein and the encoding nucleic acids.

### CROSS-REFERENCES TO RELATED APPLICATIONS

This application claims the priority benefit under 35 U.S.C. §119(e) of U.S. Provisional Application Ser. No. 61/742,882, entitled “DNA LIBRARIES ENCODING FRAMEWORKS WITH SYNTHETIC CDR REGIONS” filed Aug. 21, 2012, and U.S. Provisional Application Ser. No. 61/742,883, entitled “COMPLEX OF NON-COVALENTLY BOUND PROTEIN WITH ENCODING NUCLEIC ACIDS AND USES THEREOF” filed Aug. 21, 2012, and Non-Provisional application Ser. No. 13/971,184, entitled “DNA LIBRARIES ENCODING FRAMEWORKS WITH SYNTHETIC CDR REGIONS” and filed on Aug. 20, 2013, all of which are incorporated herein by reference in their entirety.

### BACKGROUND OF THE INVENTION

1. Field of the Invention

The present invention is related to the field of biotechnology. More specifically, the invention is directed to single domain antibody development, synthesis, and methods of use.

2Background of the Prior Art

A number of methods have been devised to identify protein-protein interactions that also allow recovery of genetic material that encodes the identified proteins. Some of these technologies work by in vivo gene expression while others utilize in vitro binding assays to identify physical interactions. Among these are the two-hybrid system, phage display and ribosome display.

An additional in vitro technology is mRNA display. Traditional mRNA display methods require continuous covalent bonds from the protein to the encoding RNA or DNA, usually by way of a puromycin-containing linker. Various methods are used to accomplish this series of continuous covalent bonds. In a first approach described in Patents U.S. Pat. No. 6,281,344, U.S. Pat. No. 6,261,804, U.S. Pat. No. 6,258,558 and U.S. Pat. No. 7,270,950, bonding is accomplished by hybridization of a linker DNA oligomer that is complementary to both the 3′ sequence of the encoding RNA strand and the 5′ sequence of a DNA oligomer that is terminated by a puromycin peptide acceptor. Covalent bonding between the RNA and DNA-puromycin oligomer is achieved, in this case, by ligation using DNA ligase. Covalent bonding between the RNA-DNA-puromycin complex and the protein is achieved, in this case, by incorporation of the puromycin at the carboxyl terminus of the nascent polypeptide during in vitro translation. U.S. Pat. No. 6,261,804 further includes post-translational incubation in high salt concentrations to improve efficiency of the puromycin incorporation process. U.S. Pat. No. 6,258,558 further describes using the nucleic acid-protein fusion to select protein-binding molecules. All of these procedures require continuous covalent bonds between the protein and the protein-encoding nucleic acid.

Other approaches to ligate the peptide acceptor to the protein-encoding nucleic acids are described in U.S. Pat. No. 6,429,300. One method described in this patent (to affix a peptide acceptor to the protein-encoding polynucleotide) uses a DNA-puromycin linker/oligomer that forms a hairpin structure that can bind to both itself and the mRNA molecule and aligns the 3′ end of the mRNA with the 5′ end of the DNA linker/oligomer. T4 DNA ligase is used to covalently attach the mRNA to the DNA-puromycin linker/oligomer. Chemical ligation methods include the use of a psoralen molecule cross-linked to the RNA molecule using UV irradiation. Other means for forming a covalent bond between a peptide acceptor and the encoding nucleotides are described. U.S. Pat. No. 6,623,926 provides other methods for chemically conjugating nucleic acids and proteins. All of the methods described in prior patents are methods to form continuous covalent bond linkages between a peptide acceptor, usually puromycin, and protein-encoding nucleotides. No methods are described to non-covalently bind the peptide acceptor with protein-encoding nucleic acid sequences for use in mRNA display procedures. U.S. Pat. No. 7,790,421 discloses another method to covalently link a protein to its encoding RNA.

U.S. Pat. No. 6,416,950 describes an mRNA display procedure using DNA-protein fusions instead of RNA-protein fusions. This patent describes a nucleic acid reverse transcription primer that is covalently bound to a peptide acceptor, typically puromycin. In a second part of the process the RNA is translated to produce a protein product which is covalently bound to the reverse transcription primer. The RNA is then reverse transcribed to produce a DNA protein fusion. The method described in this patent requires covalent bonds from the peptide acceptor to the protein encoding nucleic acids. It does not describe a process wherein a non-covalent bonds link the peptide acceptor to protein-encoding nucleic acids.

U.S. Pat. No. 6,518,018 provides an example of the use of mRNA display to select antibodies that specifically bind to desired targets. The patent claims a molecule comprising a ribonucleic acid covalently bonded through an amide bond to an antibody, wherein said antibody is encoded by said ribonucleic acid. Again covalent bonds are required for this process.

U.S. Pat. No. 6,602,685 further provides means to identify the binding of a library of polynucleotide-protein molecules with a library of solid phase bound molecule also providing a means to identify solid phase bound molecules that interact with molecules of the polynucleotide-protein molecule library.

Efficiency of the mRNA display process is improved by providing a pause sequence of the 3′ end of the encoding RNA. U.S. Pat. No. 6,214,553 claims a library of protein-encoding RNA molecules, said RNA molecules being covalently bonded at their 3′ ends to a non-RNA pause sequence. Both DNA sequences and polyethylene glycol were used as examples of pause sequences. Again, covalent bonding is a requirement.

### SUMMARY OF THE INVENTION

A method of linking a protein with its encoding nucleic acid is disclosed wherein the method of linking is non-covalent at one or more locations between the protein and the encoding nucleic acids.

This invention provides a method for non-covalently joining a protein with its encoding nucleic acid using peptide nucleic acids (PNA) to allow selection and identification of proteins with desired qualities. The invention is an improvement of a previously described technique called mRNA display. The improvement is a simplification of the procedure, eliminating the need for covalent binding of the encoding nucleic acid with the polymer binding to the encoded protein. The polymer is typically an oligonucleotide terminated at the 3′ end with a peptide acceptor such as a puromycin molecule but it can also contain organic polymer sequences such as polyethylene glycol and other peptide acceptors known in the prior art. See e.g., U.S. Pat. No. 7,790,421 incorporated herein by reference in its entirety. Compared to in vivo protein/display techniques, mixtures of molecules made by mRNA display have the potential to contain much larger libraries of molecules that are different in sequence because mRNA display is an in vitro technology.

This invention provides a method of developing a molecule that can target and bind to selected proteins or other target molecules while simultaneously carrying polynucleotide sequences that encode said targeting molecule.

In the present invention, non-covalent coupling is accomplished using peptide nucleic acid oligomers to link mRNA with a puromycin-terminated oligonucleotide. The use of normal DNA sequences as a linker results in hybridization that is too weak to survive the mRNA display procedure. When DNA based linkers are used, covalent bonding is required to provide a linkage that is strong enough to survive the procedure. The use of PNA oligomers surprisingly provides strong enough hybridization that covalent bonding is not required and the non-covalent PNA linkage is strong enough to survive the mRNA display procedure.

In one aspect of the invention, selection of desired proteins is done in iterative cycles using the following sequence of events: 1) PCR synthesis and assembly of the library, 2) mRNA in vitro transcription, 3) hybridization of at least one in vitro transcribed mRNA to a peptide nucleic acid linker/oligomer, said peptide nucleic acid linker/oligomer also hybridizing to an oligonucleotide that is terminated at the 3′ end with puromycin wherein binding among hybridized molecules is non-covalent, 4) in vitro translation with puromycin incorporation to create protein/mRNA complexes, 5) optionally binding to non-target molecules to remove unwanted binding proteins, 6) binding to target molecules, 7) recovery of bound protein/mRNA complexes, and 8) RT-PCR amplification of recovered RNA.

In one aspect of the invention, selection of desired proteins is done in iterative cycles using the following sequence of events: 1) PCR synthesis and assembly of the library, 2) mRNA in vitro transcription, 3) hybridization of at least one in vitro transcribed mRNA to a peptide nucleic acid linker/oligomer, said peptide nucleic acid linker/oligomer also hybridizing to an oligonucleotide that is terminated at the 3′ end with puromycin wherein binding among hybridized molecules is non-covalent, 4) in vitro translation with puromycin incorporation to create protein/mRNA complexes, 5) reverse transcription of mRNA to make protein/cDNA complexes, 6) optionally binding to non-target molecules to remove unwanted binding proteins, 7) binding to target molecules, 8) recovery of bound protein/cDNA complexes, and 9) PCR amplification of recovered DNA.

One aspect of the invention is a method of non-covalently joining a protein to its encoding nucleic acid comprised of the steps of: 1) selecting a DNA library containing at a minimum encoding sequences, 2) adding a promoter sequence if not present in the original sequence, 3) preparing mRNA from said DNA library using in vitro transcription, 4) hybridizing said in vitro transcribed mRNA to a peptide nucleic acid oligomer and also hybridizing said peptide nucleic acid oligomer to an oligonucleotide that is terminated at the 3′ end with a peptide acceptor such as puromycin, and 5) in vitro translating said hybridized mRNA to create a library of protein/mRNA complexes wherein binding of said hybridized molecules is non-covalent. A member of the library of protein/mRNA complexes can be selected by the steps of: binding member(s) of said library to a target molecule, recovery of bound protein/mRNA complexes, and RT-PCR amplification of recovered RNA.

### BRIEF DESCRIPTION OF THE DRAWING

FIG. 1 depicts DNA and PNA linker/oligomers for assembly of non-covalent mRNA display complexes

FIG. 2A shows a prior art method of performing mRNA display wherein the mRNA and the puromycin linker/oligomer are covalently bound using DNA ligase and a DNA oligomer hybridizing to the mRNA and the puromycin linker/oligomer.

FIG. 2B shows the present invention wherein the mRNA and the puromycin linker/oligomer are not covalently bound and the DNA oligomer is replaced with a PNA oligomer having higher hybridization strength than the corresponding DNA oligomer.

FIG. 2C shows the present invention wherein the mRNA and the puromycin linker/oligomer are not covalently bound and the DNA oligomer is replaced with a PNA oligomer having higher hybridization strength than the corresponding DNA oligomer. A photocleavable biotin is added to the 5′ end of the puromycin linker/oligomer to aid in purification during a step in the processing.

FIG. 3 shows a schematic of the mRNA display process of the present invention showing the PNA oligomer and including selection.

FIG. 4 shows a gel electrophoresed cDNA that was recovered from VHH domain immunoprecipitations following translation of VHH library using various mRNA complex conformations (see Table 1 for sample descriptions)

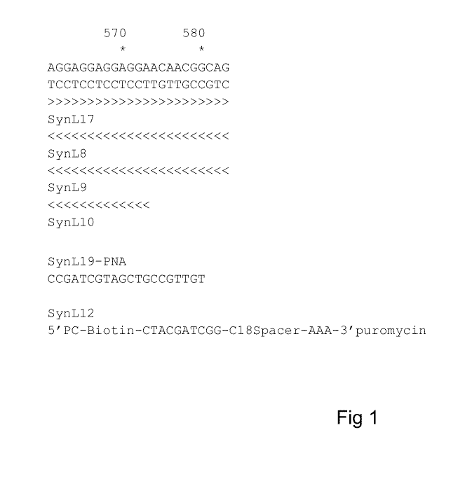

### DETAILED DESCRIPTION

The invention summarized above may be better understood by referring to the following description, drawings, and claims. This description of an embodiment, set out below to enable one to practice an implementation of the invention, is not intended to limit the preferred embodiment, but to serve as a particular example thereof. Those skilled in the art should appreciate that they may readily use the conception and specific embodiments disclosed as a basis for modifying or designing other methods and systems for carrying out the same purposes of the present invention. Those skilled in the art should also realize that such equivalent assemblies do not depart from the spirit and scope of the invention in its broadest form.

Described is a method to develop a molecule that can target and bind to selected proteins while simultaneously carrying polynucleotide sequences encoding said protein. As utilized herein, the term “ligand” means any molecule that is capable of binding other molecules. A ligand includes receptors, antibodies, VHH fragments, enzymes, or any protein that binds to another protein. The ligand may use a synthetic polypeptide that can be selected from a molecular library that is much larger than previously available. As used herein, the term “bind” or “binding” refers to the ability of a ligand to attach to its target molecule through non-covalent interactions. One embodiment provides a modified form of mRNA display in which at least one non-covalent link binds a protein or peptide to its encoding polynucleotide.

mRNA display is a technique where, in prior art methods, a nascent polypeptide is covalently linked to its encoding mRNA (Wang and Liu 2011). This linkage allows for the disassembly of the ribosome which gives the nascent polypeptide more freedom to bind to target proteins. The covalent linkage between the mRNA and polypeptide chain is typically achieved by engineering using a linker/oligomer that hybridizes to the mRNA and simultaneously hybridizes to an oligomer that contains a puromycin molecule or any other peptide acceptor. As utilized herein, a “peptide acceptor” means a molecule that is incorporated into the nascent polypeptide chain during translation by the ribosome. In prior art methods, puromycin linker/oligomers have been covalently linked by either ligation using ligase or by chemically crosslinking a modified DNA oligonucleotide to the 3′ end of mRNA(Roberts and Szostak 1997; Kurz, Gu, and Lohse 2000). The mRNA-protein complex is used to pan for binding against an immobilized target. Bound mRNA is converted to cDNA for subsequent rounds of selection and identification.

A “peptide acceptor” means any molecule capable of being added to the C-terminus of a growing protein chain by the catalytic activity of the ribosomal peptidyl transferase function. Typically, such molecules contain (i) a nucleotide or nucleotide-like moiety, for example adenosine or an adenosine analog (di-methylation at the N-6 amino position is acceptable), (ii) an amino acid or amino acid-like moiety, such as any of the 20 D- or L-amino acids or any amino acid analog thereof including O-methyl tyrosine or any of the analogs described by Ellman et al. (Meth. Enzymol. 202:301, 1991), and (iii) a linkage between the two (for example, an ester, amide, or ketone linkage at the 3′ position or, less preferably, the 2′ position). Preferably, this linkage does not significantly perturb the pucker of the ring from the natural ribonucleotide conformation. Peptide acceptors may also possess a nucleophile, which may be, without limitation, an amino group, a hydroxyl group, or a sulfhydryl group. In addition, peptide acceptors may be composed of nucleotide mimetics, amino acid mimetics, or mimetics of the combined nucleotide-amino acid structure. See U.S. Pat. No. 7,790,421.

Other possible choices for peptide acceptors include tRNA-like structures at the 3′ end of the RNA, as well as other compounds that act in a manner similar to puromycin. Such compounds include, without limitation, any compound which possesses an amino acid linked to an adenine or an adenine-like compound, such as the amino acid nucleotides, phenylalanyl-adenosine (A-Phe), tyrosyl adenosine (A-Tyr), and alanyl adenosine (A-Ala), as well as amide-linked structures, such as phenylalanyl 3′ deoxy 3′ amino adenosine, alanyl 3′ deoxy 3′ amino adenosine, and tyrosyl 3′ deoxy 3′ amino adenosine; in any of these compounds, any of the naturally-occurring L-amino acids or their analogs may be utilized. In addition, a combined tRNA-like 3′ structure-puromycin conjugate may also be used in the invention.

In the present invention, peptide nucleic acids replace the DNA oligomer used in the prior art as a method to link encoding RNA to a puromycin terminated oligomer. Peptide nucleic acids (PNA) were originally described by Nielsen et al. in 1991(Nielsen et al. 1991). Peptide nucleic acids are DNA analogs in which an N-(2-aminoethyl)glycine polyamide replaces the phosphate-ribose ring backbone, and a methylene-carbonyl linker connects nucleo-bases to the central amine of N-(2-aminoethyl)glycine(Kim et al. 2008). DNA and RNA have a deoxyribose and ribose sugar backbone, respectively, whereas PNA's backbone is composed of repeating N-(2-aminoethyl)-glycine units linked by peptide bonds. The various purine and pyrimidine bases are linked to the backbone by methylene carbonyl bonds. PNAs are described like peptides, with the N-terminus at the first (left) position and the C-terminus at the right.

PNAs hybridize to DNA, RNA and other PNA sequences following Watson-Crick base pairing rules (Egholm et al. 1993). Binding of PNA to DNA or RNA has higher affinity than DNA/DNA or DNA/RNA binding as shown by the higher melting temperatures of duplexes containing PNA(Nielsen et al. 1991). The increased melting temperatures are thought to be a result of reduced charge in the PNA backbone which in turn reduces charge repulsion seen in DNA/DNA or DNA/RNA duplexes. In addition, the base pairing is more sensitive to mismatching than DNA/DNA or a DNA/RNA structures making the binding more specific. PNA/DNA hybridizations are also less sensitive to high salt concentrations than are corresponding DNA/DNA hybridizations(Tomac et al. 1996). PNA can hybridize to RNA or DNA in at least two forms. One is a simple hybridization with one PNA oligomer. Another form is a triplex with two PNA oligomers. The preferred form for the current invention is hybridization with one PNA oligomer because of its simplicity. In the prior art triplex formation was described as a method to link RNA to a peptide acceptor terminated oligonucleotide. While this method works it requires additional expensive reagents. One embodiment of the present invention, describes a method for the use of single PNA hybridization under specific conditions that avoids the need of triplex formation described in the prior art.

Throughout this discussion, the PNA oligomer, the DNA equivalent of the PNA oligomer, and the puromycin terminated oligomer may be referred to as either linkers or oligomers or linker/oligomers. Thus, in these contexts the terms “oligomer” and “linker” are equivalent.

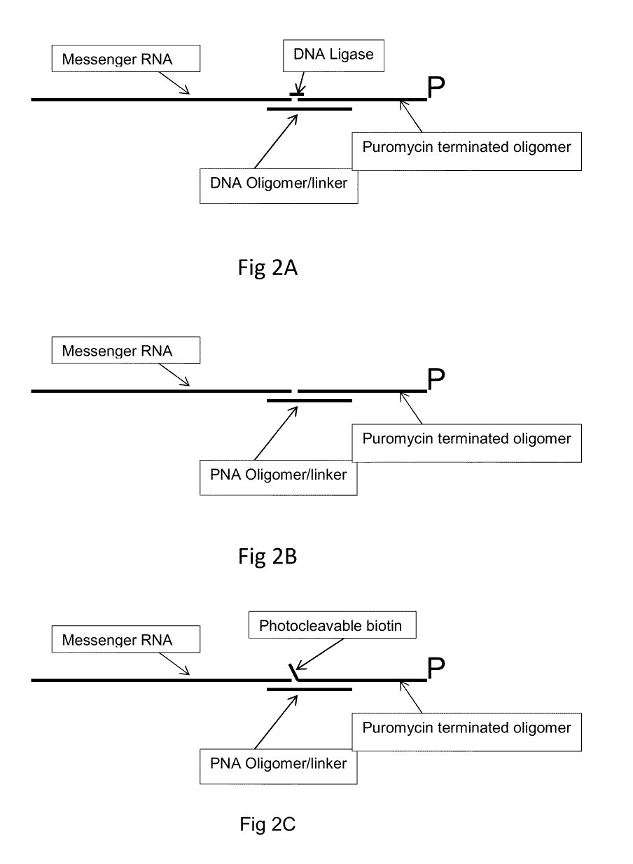

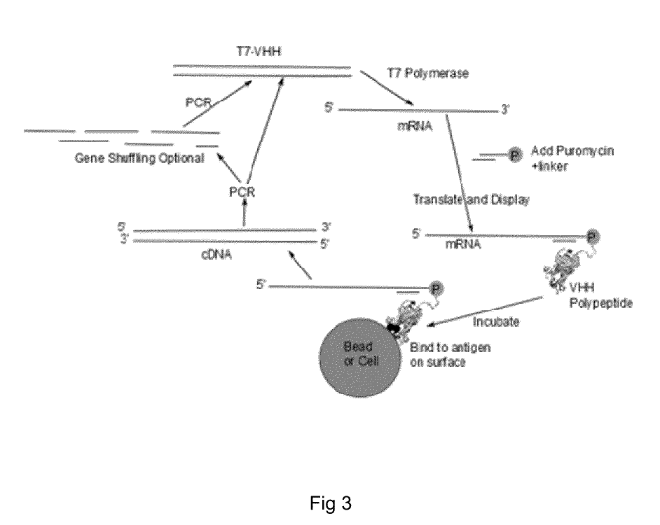

This invention provides a simplified method for non-covalently joining a protein with its encoding nucleic acid using PNA to allow selection and identification of proteins with desired qualities. The non-covalent binding can be at one or more locations between the protein and its encoding polynucleotide (RNA or DNA). FIG. 2A shows a prior art method of performing mRNA display wherein the mRNA and a puromycin-terminated linker are covalently joined together using DNA ligase while a DNA oligomer transiently holds the mRNA and puromycin linker/oligomer in close proximity by hybridizing to both molecules. Non-covalent hybridization of the DNA oligomer alone is too weak to maintain association of the mRNA and the puromycin linker throughout the mRNA display process. FIG. 2B shows one embodiment of the present invention wherein the mRNA and the puromycin linker are not covalently bound and the DNA oligomer is replaced with a PNA oligomer that has higher hybridization strength than the corresponding DNA oligomer. FIG. 2C shows one embodiment of the present invention wherein the mRNA and the puromycin linker are not covalently bound and the DNA oligomer is replaced with a single PNA oligomer having higher hybridization strength than the corresponding DNA oligomer. In FIG. 2C, a photocleavable biotin is added to the 5′ end of the puromycin linker to aid in purification during a step in the processing. In the present invention, the higher hybridization strength of the PNA linker/oligomer assures association of the puromycin linker and the mRNA without requiring the formation of covalent bonds between any of the three associated molecules. Additionally, after in vitro transcription, the higher bonding strength of the PNA oligomer allows the non-covalent binding of the encoding mRNA, with or without cDNA, to continue to maintain association of the protein with its encoding polynucleotides throughout subsequent processing.

The invention is an improvement of a previously described mRNA display technique. The improvement is a simplification of the procedure, eliminating the need for covalent binding of the encoding nucleic acid with the polymer that binds to the encoded protein. The polymer is typically an oligonucleotide terminated at the 3′ end with a peptide acceptor such as a puromycin molecule but it can also contain organic polymer sequences such as polyethylene glycol. Compared to in vivo protein/display techniques, mixtures of molecules made by mRNA display have the potential to contain much larger libraries of molecules that are different in sequence because mRNA display is an in vitro technology.

In the present invention, mRNA display selection techniques may be used to isolate molecules that bind to immobilized proteins, bind to soluble proteins followed by immunoprecipitation, or bind to proteins on cells and simultaneously recover the polynucleotide that encodes the binding molecules.

In one aspect of the invention, selection of desired proteins is done in iterative cycles using the following sequence of events: 1) PCR synthesis and assembly of the library, 2) mRNA in vitro transcription, 3) hybridization of at least one in vitro transcribed mRNA to a single peptide nucleic acid linker/oligomer, said peptide nucleic acid linker/oligomer also hybridizing to an oligonucleotide that is terminated at the 3′ end with a peptide acceptor molecule, typically puromycin wherein binding among hybridized molecules is non-covalent, 4) in vitro translation with puromycin incorporation to create protein/mRNA complexes, 5) binding to target molecules, 6) recovery of bound protein/mRNA complexes by washing away or removing non-bound mRNA display product, and 7) RT-PCR amplification of recovered RNA. Optionally, after step 4 above but before step 5, the resulting protein/mRNA complex mixture can be incubated with non-target molecules to remove unwanted mRNA display product binding to non-target proteins.

In a preferred embodiment, the hybridization of the mRNA/PNA/Puromycin terminated linker complexes in step 3 of the previous paragraph are made by combining 1:1:1, 1:1.1:1.1, 1:2:2, 1:5:5 or other molar ratios of mRNA:PNA:puromycin terminated linker. Preferably, the PNA and puromycin terminated linker are mixed first and given sufficient time to allow hybridization of the PNA and puromycin terminated linker. The mRNA is then added and hybridizes to the pre-formed PNA-Puromycin terminated linker. This method prevents loss of product as a result of forming RNA-PNA hybridizations that cannot bind to PNA-Puromycin terminated linker hybridizations. The amount of final product, mRNA:PNA:puromycin terminated linker is thus improved.

In another aspect of the invention, a method of non-covalently joining a protein with its encoding nucleic acid is described comprised of the steps of; selecting a DNA library containing at a minimum encoding sequences; adding a promoter sequence if not present in the original sequence; preparing mRNA from said DNA library using in vitro transcription; hybridizing said in vitro transcribed mRNA to a peptide nucleic acid linker/oligomer, said peptide nucleic acid linker/oligomeroligomer also hybridizing to an oligonucleotide that is terminated at the 3′ end with a peptide acceptor such as puromycin; in vitro translating said hybridized mRNA to create a library of protein/mRNA complexes wherein binding of said hybridized molecules is non-covalent.

In one aspect of the invention, selection of desired proteins is done in iterative cycles using the following sequence of events: 1) PCR synthesis and assembly of a library with protein encoding sequences and other sequences such as promoter sequences and linker/oligomer sequences as needed to do the subsequent steps of the procedure, 2) mRNA in vitro transcription, 3) hybridization of at least one in vitro transcribed mRNA to a single peptide nucleic acid linker/oligomer, said peptide nucleic acid linker/oligomer also hybridizing to an oligonucleotide that is terminated at the 3′ end with puromycin wherein binding among hybridized molecules is non-covalent, 4) in vitro translation with puromycin incorporation to create protein/mRNA complexes, 5) reverse transcription of mRNA to make protein/cDNA complexes, 6) optionally binding to non-target molecules to remove unwanted binding proteins, 7) binding to target molecules, 8) recovery of bound protein/cDNA complexes, and 9) PCR amplification of recovered DNA.

In one aspect of the invention, selection of desired proteins is done in iterative cycles using the following sequence of events: 1) synthesis of a DNA library comprised of backbone sequences plus synthetic variable regions, 2) PCR synthesis and assembly of a library with protein encoding sequences and other sequences such as promoter sequences and linker/oligomer sequences as needed to do the subsequent steps of the procedure, 3) mRNA in vitro transcription, 4) hybridization of at least one in vitro transcribed mRNA to a single peptide nucleic acid linker/oligomer, said peptide nucleic acid linker/oligomer also hybridizing to an oligonucleotide that is terminated at the 3′ end with puromycin wherein binding among hybridized molecules is non-covalent, 5) in vitro translation with puromycin incorporation to create protein/mRNA complexes, 6) reverse transcription of mRNA to make protein/cDNA complexes, 7) optionally binding to non-target molecules to remove unwanted binding proteins, 8) binding to target molecules, 9) recovery of bound protein/cDNA complexes, and 9) PCR amplification of recovered DNA.

In one aspect of the invention, selection of desired proteins is done in iterative cycles using the following sequence of events: 1) synthesis of a DNA library comprised of backbone sequences plus synthetic variable regions, the synthetic variable regions being synthesized using random trimer phosphoramidites, 2) PCR synthesis and assembly of a library with protein encoding sequences and other sequences such as promoter sequences and linker/oligomer sequences as needed to do the subsequent steps of the procedure, 3) mRNA in vitro transcription, 4) hybridization of at least one in vitro transcribed mRNA to a single peptide nucleic acid linker/oligomer, said peptide nucleic acid linker/oligomer also hybridizing to an oligonucleotide that is terminated at the 3′ end with puromycin wherein binding among hybridized molecules is non-covalent, 5) in vitro translation with puromycin incorporation to create protein/mRNA complexes, 6) reverse transcription of mRNA to make protein/cDNA complexes, 7) optionally binding to non-target molecules to remove unwanted binding proteins, 8) binding to molecules, 9) recovery of bound protein/cDNA complexes, and 9) PCR amplification of recovered DNA.

In one aspect of the invention, mRNA display ligands may be selected using affinity protein binding techniques. In one example, approximately six iterations of the process described below may be needed to isolate high affinity binding members of a library containing cancer specific ligands: Camelid VHH library DNA may be transcribed to mRNA using commercially available in vitro transcription kits. A puromycin-conjugated DNA oligonucleotide may be attached to the 3′ end of the mRNA molecules via a high-affinity peptide nucleic acid (PNA) linker/oligomer molecule, the high affinity PNA molecule allowing easy and efficient binding of a DNA-puromycin linker/oligomer (with an optional photocleavable biotin) without the need for covalent modification to the mRNA. The mRNA/PNA/DNA-puromycin complexes may be used to program rabbit reticulocyte lysates for in vitro translation. Nascent proteins translated from the mRNA may become attached to the mRNA complex by virtue of the puromycin molecule in the DNA-puromycin linker/oligomer. First-strand cDNA synthesis may be carried out at this point to help protect the mRNA as a RNA/DNA duplex. Protein/mRNA complexes may be isolated and purified on paramagnetic streptavidin beads by virtue of the optional photocleavable biotin moiety. Paramagnetic beads are particles that can be isolated from a liquid by exposure to a magnetic field.

The purified protein/mRNA complexes may be used to pan for binding to target molecules. Both positive and negative selections may be used to identify binding proteins that are specific to the target molecules. After panning, the encoding polynucleotide may be recovered using reverse transcriptase polymerase chain reaction (RT-PCR) if the polynucleotide is messenger RNA or by polymerase chain reaction (PCR) if first strand cDNA synthesis was performed prior to panning

### EXAMPLE 1

### One Method for Assembling Non-Covalent RNA-Protein Complexes for mRNA Display Selection

Step 1. mRNA is transcribed in 40 μl reactions using a commercially available T7 transcription kit (i.e. MEGAscript, Life Technologies) from 0.5-1 μg of synthetic camelid VHH library DNA that contain random variable CDR sequences that are generated by random incorporation of phosphoramidite trimers. Template DNA is removed by DNase digestion and RNA is recovered by phenol/chloroform extraction plus ethanol precipitation. The recovered mRNA is quantified by photospectrometry and diluted to a concentration of 1-5 mg/ml in nuclease-free water. mRNA from multiple transcription reactions may be pooled to increase the diversity of the mRNA library.

Step 2. A PNA oligonucleotide (SynL19-PNA (SEQ ID No. 3)) is synthesized that contains sequences that can simultaneously anneal to the 3′ end of the camelid VHH antibody mRNA (SynL17 (SEQ ID No. 2)) and a modified DNA-puromycin linker/oligomer (SynL12 (SEQ ID No. 1)). Both synthetic constructs can be synthesized by appropriate commercial vendors. The modified DNA-puromycin linker/oligomer can be synthesized to contain a 5′ photocleavable biotin, an annealing sequence, and a 3′ puromycin that is separated from the annealing sequence by an 18-carbon spacer. Stock solutions of the SynL19-PNA (SEQ ID No. 3) and SynL12 (SEQ ID No. 1) are made by dissolving each to 100-500 μM in nuclease-free water.

Step 3. mRNA/Syn19-PNA/SynL12 (SEQ ID No. 1) complexes are made by combining 1:1:1, 1:2:1, 1:5:5 or other molar ratios of mRNA:SynL19-PNA (SEQ ID No. 3):SynL12 (SEQ ID No. 1) in nuclease-free water so that the mRNA is at a final concentration of 1 μg/μl. In a preferred embodiment, the molecules are mixed in the order of PNA, puromycin terminated linker, and then mRNA with an incubation before adding the mRNA sufficient to allow hybridization of the PNA and puromycin terminated linker. The mixture is incubated at 25° C. for at least 10 min. Alternatively, the mixture is heated to 95° C. for 1 min, then incubated at 55° C. for 3 minutes prior to incubation at 25° C.

Step 4. Five microliters of the mRNA/SynL19-PNA (SEQ ID No. 3) /SynL12 (SEQ ID No. 1) complex (5 μg mRNA) are used in 25 μl reactions to program commercially available reticulocyte lysates (i.e. Retic Lysate IVT, Life Technologies) for in vitro translation. Multiple reactions may be pooled to increase library diversity. Translation of the mRNA/SynL19-PNA (SEQ ID No. 3) /SynL12 (SEQ ID No. 1) complex results in the incorporation of nascent polypeptides into the complex via the puromycin moiety. The protein is covalently attached to the DNA-puromycin linker/oligomer, but non-covalently bound to the camelid VHH antibody mRNA. The mRNA/SynL19-PNA (SEQ ID No. 3) /SynL12 (SEQ ID No. 1) /protein complex may be used directly in binding assays. Alternatively, the complex may be purified from the reticulocyte lysate by virtue of the photocleavable biotin moiety on the SynL12 (SEQ ID No. 1) DNA-puromycin linker/oligomer. Complexes are bound to magnetic streptavidin beads and washed to remove reticulocyte lysate, Bound complexes are then released from the magnetic beads by exposure to UV light.

Substrates for affinity binding and antibody selection may be from several sources. In one example, human cancer cell lines and tissue sections from human cancer and other human tissues can be used. Human cell lines other than selected cancer cells and non-cancer tissues may be used for negative selections to remove antibodies that bind to non-target tissue or cells. After isolating several of these antibodies, cDNAs encoding the antibodies may be cloned and sequenced to determine the diversity of the antibodies selected by this technique. Individual candidate antibodies may be purified and used in immunohistochemistry studies to identify where the antibodies bind within the target tissue. Antibodies that bind to targeted cells in tissue (in one example prostate cancer and normal prostate cells) may be retained while those that bind to non-target cells (for example epithelial cells, endothelial cells and fibroblasts) may be discarded or kept in a separate library. Any cancer cell line, non-cancer cell line, or thin or thick sections of tissues may be substituted for the prostate cancer cells in this example. Any cancerous or normal cells or tissues from any species to include human may be used.

Since RNase is ubiquitous in live cells, RNase inhibitors must be used when using cells or tissues as solid substrates. Commercially available protein based RNase inhibitors are known and can be used. Some small molecule chemicals have RNase inhibiting activity. Each of the following chemicals has been demonstrated to inhibit RNase activity. In some cases, the inhibitory effects are permanent (i.e. the inhibitor can be removed after treatment). In other cases, the inhibitor must be present to exert its effects: Vanadyl-ribonucleoside(Lindquist, Lynn, and Lienhard 1973); Oligovinylsulfonic acid(Smith, Soellner, and Raines 2003); Polyvinylsulfonic acid(Smith, Soellner, and Raines 2003); Iodoacetate(Harada and Irie 1973); Bromoacetate(Harada and Irie 1973); Aurin tricarboxylic acid(Ghosh, Giri, and Bhattacharyya 2009); 5′ diphosphoadenosine 3′ phosphate(Russo, Shapiro, and Vallee 1997); 5′ diphosphoadenosine 2′ phosphate(Russo, Shapiro, and Vallee 1997); Diribonucleoside 2′,5′ monophosphates(White, Bauer, and Lapidot 1977); Diribonucleoside 3′,5′ monophosphates(White, Bauer, and Lapidot 1977); Guanylyl 2′, and 5 ′ guanosine(White, Rapoport, and Lapidot 1977; Koepke et al. 1989).

Another substrate used for selection is solid phase target molecules that are bound to the surfaces of microtiter plates or microbeads. Target molecules (peptides, proteins, polysaccharides, membrane fragments and other molecules) can be non-covalently adhered to or covalently linked to commercially available plasticware (such as 96 well ELISA plates) or to a variety of commercially available magnetic or non-magnetic microbeads such as those available from Bangs Labs using known covalent binding methods.

In one example of binding methods, peptides can be synthesized with the target peptide sequence plus additional linker/oligomer amino acids and an N-terminal cysteine that is used for crosslinking to amine coated magnetic beads using heterobifunctional NHS-maleimide-mediated conjugation. Two peptides with different linker/oligomer peptides can be made to eliminate selection of ligands that bind to the linker/oligomer. A separate PEG (polyethylene glycol) linker/oligomer may be first bound to the solid surface to limit steric hindrance. The amine-PEG-Carboxyl linker may be attached to carboxyl coated beads using standard carbodiimide-NHS chemistry.

In another example, some of the peptides may be directly bound to carboxyl coated or PEG-carboxyl coated beads using carbodiimide-NHS chemistry. A negative control magnetic bead can be made with linkers only, or other negative molecules to include peptides with single amino acid substitutes.

If using whole or partial proteins, they can be bound to solid surfaces by adhesion or using known covalent binding techniques. As an example, proteins can be directly bound by their amines to carboxyl coated beads using standard two-step carbodiimide-NHS chemistry.

In addition to solid surfaces, selection also can be done by immunoprecipitation.

This is especially useful if a known second antibody is already available. For immunoprecipitation, mRNA display product consisting of linked protein and its encoding polypeptide (mRNA or cDNA) is immunoprecipitated by a second antibody recognizing the target protein or affinity tags expressed with the target protein.

### EXAMPLE 2

Demonstration that non-covalent linkage of the present invention maintains association of a protein and its encoding polynucleotide through immunoprecipitation and recovery of cDNA: A DNA library of camelid VHH fragments was prepared according to a method described in application Ser. No. 13/971,184 which is hereby incorporated by reference in its entirety. The DNA library was transcribed into mRNA using a commercial T7 transcription kit. The mRNA was used to make complexes with a puromycin linker/oligomer in various conformations (see Table 1). Covalent vs. non-covalent linkages between the VHH mRNA were prepared by ligating the mRNA to the puromycin linker/oligomer or leaving ligase out of the reaction. Sample Number 4 represents the prior art where a linker/oligomer and ligase are both required to make a stable molecule that is covalently linked from the protein to its encoding polynucleotides.

TABLE 1
Experimental design to compare non-covalent vs. covalent mRNA
complexes in mRNA display

| | | PNA Coupler | DNA coupler | | |
|---|---|---|---|---|---|
| Sample | | (SYNL19-PNA, | (SYNL19-DNA, | Puromycin | |
| Number | VHH mRNA | SEQ ID No. 3) | SEQ ID No. 4) | Linker/oligomer | Ligase |
| 1 | + | + | − | + | − |
| 2 | + | + | − | + | + |
| 3 | + | − | + | + | − |
| 4 | + | − | + | + | + |

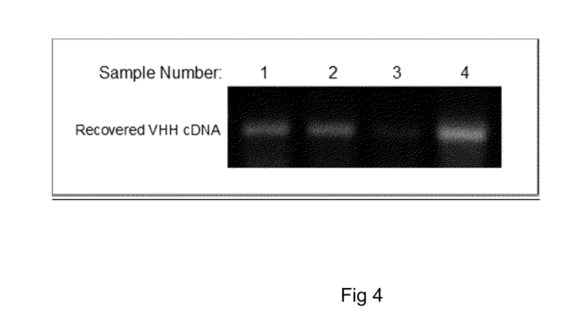

The VHH mRNA complexes were used to program wheat germ extracts for in vitro translation, then the translation mixtures were further processed to maximize mRNA-puromycin-protein complex formation. Anti-FLAG™ antibody plus protein-G paramagnetic beads were used to immunoprecipitate translated VHH domain protein from the translation reaction mixtures. After extensive washing, VHH cDNA was recovered from the beads by RT-PCR, and then visualized on agarose gels. Recovery of cDNA in this experiment means that the protein remained linked to its encoding polynucleotide throughout the process because all non-bound molecules were removed in the washing step and the RNA has no means to bind other than through the linked protein encoded by that RNA. FIG. 4 shows an agarose gel electrophoresis showing that equivalent amounts of cDNA are recovered from reactions that contain the non-covalent linker/oligomer without (SYNL19-PNA, SEQ ID No. 3) (lane 1) or with (lane 2) covalent ligation of the RNA to the linker/oligomer. In comparison, very little cDNA is recovered when a prior art DNA coupler (SYNL19-DNA, SEQ ID No. 4) is used without ligation (lane 3) of the RNA to the linker/oligomer and cDNA recovery was only seen for the prior art configuration when ligase was used (lane 4). These data indicate that the non-covalent process of this invention is superior to the prior art process because the process of this invention works without the requirement of covalent linkage.

Examples described above are only some of the methods of performing mRNA display using PNA oligomers to eliminate the need for covalent binding of the puromycin linker/oligomer to its encoding mRNA. The examples for making camelid antibodies using synthetic CDR regions or other ligands are similarly not limiting. None of these examples are meant to be limiting.

The invention has been described with references to a preferred embodiment. While specific values, relationships, materials and steps have been set forth for purposes of describing concepts of the invention, it will be appreciated by persons skilled in the art that numerous variations and/or modifications may be made to the invention as shown in the specific embodiments without departing from the spirit or scope of the basic concepts and operating principles of the invention as broadly described. It should be recognized that, in the light of the above teachings, those skilled in the art can modify those specifics without departing from the invention taught herein. Having now fully set forth the preferred embodiments and certain modifications of the concept underlying the present invention, various other embodiments as well as certain variations and modifications of the embodiments herein shown and described will obviously occur to those skilled in the art upon becoming familiar with such underlying concept. It is intended to include all such modifications, alternatives and other embodiments insofar as they come within the scope of the appended claims or equivalents thereof. It should be understood, therefore, that the invention may be practiced otherwise than as specifically set forth herein. Consequently, the present embodiments are to be considered in all respects as illustrative and not restrictive.

### REFERENCES

The following references cited in the specification are hereby incorporated by reference in their entirety.
Egholm, M, O Buchardt, L Christensen, C Behrens, S M Freier, D A Driver, R H Berg, S K Kim, B Norden, and P E Nielsen. 1993. “PNA Hybridizes to Complementary Oligonucleotides Obeying the Watson-Crick Hydrogen-bonding Rules.” Nature 365 (6446) (October 7): 566-568. doi:10.1038/365566a0.
Ghosh, Utpal, Kalyan Giri, and Nitai P Bhattacharyya. 2009. “Interaction of Aurintricarboxylic Acid (ATA) with Four Nucleic Acid Binding Proteins DNase I, RNase A, Reverse Transcriptase and Taq Polymerase.” Spectrochimica Acta. Part A, Molecular and Biomolecular Spectroscopy 74 (5) (December): 1145-1151. doi:10.1016/j.saa.2009.09.024.
Harada, Masatomi, and Masachika Irie. 1973. “Alkylation of Ribonuclease from Aspergillus Saitoi with Iodoacetate and Iodoacetamide.” Journal of Biochemistry 73 (4) (April 1): 705-716.
Kim, Sung Kee, Hyunil Lee, Jong Chan Lim, Hoon Choi, Jae Hoon Jeon, Sang Youl Ahn, Sung Hee Lee, and Won Jun Yoon. 2008. “U.S. Pat. No. 7,371,859-PNA Monomer and Precursor.”
Koepke, J, M Maslowska, U Heinemann, and W Saenger. 1989. “Three-dimensional Structure of Ribonuclease T1 Complexed with Guanylyl-2′,5′-guanosine at 1.8 A Resolution.” Journal of Molecular Biology 206 (3) (April 5): 475-488.
Kurz, M, K Gu, and P A Lohse. 2000. “Psoralen Photo-crosslinked mRNA-puromycin Conjugates: a Novel Template for the Rapid and Facile Preparation of mRNA-protein Fusions.” Nucleic Acids Research 28 (18) (September 15): E83.
Lindquist, R N, J L Lynn Jr, and G E Lienhard. 1973. “Possible Transition-state Analogs for Ribonuclease. The Complexes of Uridine with oxovanadium(IV) Ion and vanadium(V) Ion.” Journal of the American Chemical Society 95 (26) (December 26): 8762-8768.
Nielsen, P. E., M. Egholm, R. H. Berg, and O. Buchardt. 1991. “Sequence-Selective Recognition of DNA by Strand Displacement with a Thymine-Substituted Polyamide.” Science 254 (5037) (December 6): 1497-1500. doi:10.1126/science.1962210.
Roberts, R W, and J W Szostak. 1997. “RNA-peptide Fusions for the in Vitro Selection of Peptides and Proteins.” Proceedings of the National Academy of Sciences of the United States of America 94 (23) (November 11): 12297-12302.
Russo, N, R Shapiro, and B L Vallee. 1997. “5′-Diphosphoadenosine 3′-phosphate Is a Potent Inhibitor of Bovine Pancreatic Ribonuclease A.” Biochemical and Biophysical Research Communications 231 (3) (February 24): 671-674. doi:10.1006/bbrc.1997.6167.
Smith, Bryan D., Matthew B. Soellner, and Ronald T. Raines. 2003. “Potent Inhibition of Ribonuclease A by Oligo(vinylsulfonic Acid).” Journal of Biological Chemistry 278 (23) (June 6): 20934-20938. doi:10.1074/jbc.M301852200.
Tomac, Sebastian, Munna Sarkar, Tommi Ratilainen, Pernilla Wittung, Peter E. Nielsen, Bengt Nordén, and Astrid Gräslund. 1996. “Ionic Effects on the Stability and Conformation of Peptide Nucleic Acid Complexes.” J. Am. Chem. Soc. 118 (24): 5544-5552. doi:10.1021/ja9604951.
Wang, Hui, and Rihe Liu. 2011. “Advantages of mRNA Display Selections over Other Selection Techniques for Investigation of Protein-protein Interactions.” Expert Review of Proteomics 8 (3) (June): 335-346. doi:10.1586/epr.11.15.
White, M D, S Bauer, and Y Lapidot. 1977 “Inhibition of Pancreatic Ribonuclease by 2′-5′ and 3′-5′ Oligonucleotides.” Nucleic Acids Research 4 (9) (September): 3029-3038.
White, M D, S Rapoport, and Y Lapidot. 1977. “Guanylyl 2′-5′ Guanosine as an Inhibitor of Ribonuclease T1.” Biochemical and Biophysical Research Communications 77 (3) (August 8): 1084-1087.

what is claimed is:

1. A method of non-covalently joining a protein to its encoding nucleic acid, comprising:
A. preparing mRNA from a DNA library using in vitro transcription,
B. hybridizing at least one of said in vitro transcribed mRNA to a single peptide nucleic acid (PNA) oligomer, wherein said PNA oligomer is configured for hybridization with both the mRNA and an oligonucleotide comprising a peptide acceptor, and
C. in vitro translating said hybridized mRNA to create a library of protein/mRNA complexes.

2. The method of claim 1, further comprising selecting a member of said library of protein/mRNA complexes by:
A. binding at least one member of said library to a target molecule,
B. recovering target molecule bound protein/mRNA complexes, and
C. amplifying recovered RNA from said bound protein/mRNA complex.

3. The method of claim 1, wherein said in vitro translated and hybridized mRNA is reverse transcribed to produce protein/cDNA complex library.

4. The method of claim claim 3, further comprising selecting a member of said library of protein/cDNA complexes by:
A. binding at least one member of said library to at least one target molecule,
B. recovering target molecule bound protein/cDNA complexes, and
C. amplifying DNA from said bound protein/cDNA complex.

5. The method of claim 1, wherein said peptide acceptor is on the 3′ terminus end of the oligonucleotide.

6. The method of claim 1, wherein the peptide acceptor is selected from the group consisting of puromycin, amino acid nucleotides, amide-linked nucloetides, and tRNA-like 3′ puromycin conjugates.

7. The method of claim 2, wherein said mRNA is amplified through RT-PCR.

8. The method of claim 1, further comprising adding a promoter sequence to DNA sequences in said DNA library.

9. The method of claim 1, wherein said DNA library comprises at least one encoding sequence.

10. The method of claim 4, wherein said DNA is amplified by PCR.

11. The method of claim 1, wherein the DNA library comprises sequences for single chain antibodies.

12. The method of claim 6, wherein the amino acid nucleotides are selected from the group consisting of phenylalanyl-adenosine (A-Phe), tyrosyl adenosine (A-Tyr), and alanyl adenosine (A-Ala).

13. The method of claim 6, wherein the amide-linked nucleotides are selected from the group consisting of phenylalanyl 3′ deoxy 3′ amino adenosine, alanyl 3′ deoxy 3′ amino adenosine, and tyrosyl 3′ deoxy 3′ amino adenosine.
